(19) United States
(12) Patent Application Publication
(10) Pub. No.: US 2009/0286173 A1
(43) Pub. Date: Nov. 19, 2009

(54) ELECTRONIC COMPONENT FORMING APPARATUS, ELECTRONIC COMPONENT FORMED WITH IT AND FORMING METHOD THEREOF

(76) Inventors: Kazuya Ushirokawa, Osaka (JP); Shozo Ochi, Osaka (JP)

Correspondence Address: WENDEROTH, LIND & PONACK L.L.P., 1030 15th Street, N.W., Suite 400 East, Washington, DC, 20005-1503, US

(21) Appl. No.: 12/465,847

(22) Filed: May 14, 2009

(30) Foreign Application Priority Data
May 15, 2008 (JP) 2008-128006

(51) Int. Cl. G03F 7/20; A61N 5/00

(52) U.S. Cl. 430/30; 250/492.2

(57) ABSTRACT

An electronic component forming apparatus for forming an electronic component by radiating light to a photosensitive conductive resin provided on a forming work material, comprising radiation device for radiating light to the forming work material, detection device for detecting reflecting light reflected from the forming work material, and control device for controlling the radiating light responsive to an amount of the light detected by the detection device.

### BACKGROUND OF THE INVENTION

1. Field of the Invention

The present invention relates to an electronic component forming apparatus for forming electrodes especially of the type having a protruding shape and a pattern of wiring traces by radiating light to a photosensitive conductive resin provided on a forming work material. The invention also relates to an electronic component formed by using the electronic component forming apparatus, and a forming method thereof.

2. Background Art

Efforts are being made in recent years to achieve simple processes of forming a mount substrate for mounting semiconductor elements. The use of stereo-lithography for forming an electrical insulation layer and a wiring conductor layer is one example of such method disclosed in Japanese Patent Unexamined Publication, No. 1998-112474 (hereafter referred to as “patent reference 1”). The method of forming wiring substrate taught in the patent reference 1 is outlined herein below.

First, an electrical insulation layer is formed with a liquid form insulation resin of photo-curing type by using the stereo-lithography. Next, light is radiated to a liquid form conductive rosin of photo-curing type by using the stereo-lithography to photo-cure portions of the liquid-form conductive resin that make up a pattern of wiring traces. Finally, the liquid form conductive resin other than the photo-cured portions is removed to complete the pattern of wiling traces for use as a wiling conductor layer according to this method.

There is also a method of forming protruding electrodes used for electrically connecting electronic components with a conductive resin containing a photosensitive resin and a conductive filler by using the stereo-lithography as disclosed in Japanese Patent Unexamined Publication, No. 2007-250618 (referred to as “patent reference 2”).

Referring now to FIG. 7A to FIG. 7C, description is provided hereinafter in brief of the method of forming protruding electrodes disclosed in the patent reference 2.

FIG. 7A is a schematic diagram showing a structure of an electronic component forming apparatus employing a reduced projection exposure method, FIG. 7B is a plan view showing a diagrammatic shape of a photomask used in the apparatus of FIG. 7A, and FIG. 7C is a plan view showing details of the photomask of FIG. 7B. In an instance that the electronic components are semiconductor elements, protruding electrodes may be formed on a semiconductor wafer of the type having a plurality of semiconductor elements formed on a silicon substrate.

At the outset, a semiconductor wafer having a number of semiconductor elements formed thereon, as represented by forming work material 1, is immersed in photosensitive conductive resin 2 of a liquid form contained in container 12 as shown in FIG. 7A. Provided above container 12 are light source 3, lighting optical system 9, liquid crystal mask 7 used as a photomask and reduced-projection optical system 10. Controller 6 is used to control open segments in liquid crystal mask 7 to adjust shapes of the protruding electrodes and the pattern of wiring traces in a comparatively large scale.

In the structure illustrated above, light 11 radiated from light source 3 passes through lighting optical system 9 and transmits liquid crystal mask 7. This brings a pattern formed in liquid crystal mask 7 to project on the semiconductor wafer, e.g., forming work material 1, in a size reduced by reduced-projection optical system 10.

It then causes photosensitive conductive resin 2 of the liquid form to become cured at portions where light 11 is radiated in area 14 corresponding to four semiconductor elements, for instance, as shown in FIG. 7A. FIG. 7B shows one example of the pattern formed in liquid crystal mask 7 used as the photomask. Shown here is the mask pattern for protruding electrodes of four semiconductor elements that can be formed all at once. There are a plural number of open segments 15 to form the protruding electrodes within mask area 7a corresponding to one of the semiconductor elements. These open segments 15 are provided in locations corresponding to electrode terminals around the periphery of the semiconductor element, for instance, and the protruding electrodes are formed on the electrode terminals through open segments 15.

When using the photomask of the above structure, the light is radiated while shifting the photomask at steps of every four semiconductor elements to cure the liquid form photosensitive conductive resin in the individual areas. The protruding electrodes are thus formed on the entire surface of the semiconductor wafer.

Referring now to FIG. 7C, description is provided hereinafter in brief of liquid crystal mask 7 that serves as the photomask.

FIG. 7C is an enlarged plan view showing a part of liquid crystal mask 7 used as the photomask. What is shown here is an example, in which each of open segments 15 in liquid crystal mask 7 is configured of thirty-six liquid crystal cells 16 to form one protruding electrode for the sake of simplifying the explanation.

Open segments 15, each configured of thirty-six liquid crystal cells 16 as shown in FIG. 7C, are arranged in a manner as illustrated in FIG. 7B for instance, and light 11 is radiated through these open segments 15.

The radiation of light 11 cures photosensitive conductive resin 2 on the semiconductor wafer in the positions corresponding to open segments 15 to form the protruding electrodes within area 14. Liquid crystal mask 7 is then shifted onto the next four semiconductor elements, and the light radiated in the same manner as above. The protruding electrodes are formed on the individual electrode terminals over the entire surface of the semiconductor wafer shown as forming work material 1, by repeating the above process of shifting.

When it is desired to increase a height of the protruding electrodes, the semiconductor wafer, e.g., forming work material 1 is moved down into liquid-form photosensitive conductive resin 2 by a certain extent necessary to provide a second layer of desired thickness after formation of the first layer of the protruding electrodes. The second layer is then formed under this condition by repeating the light exposure in the similar manner as discussed above. The protruding electrodes can be formed into a shape having tapered tip when open segments 15 are reduced to a size smaller than that used for the first layer by regulating liquid crystal cell 16 before making another light exposure. When thirty-six liquid crystal cells 16 are used to make each open segment for the first layer, for instance, the protruding electrodes of a frustum of pyramid shape can be formed by making light exposure through open segments, each consisting of sixteen liquid crystal cells 16 for the second layer.

There is also a method of forming the protruding electrodes unitary without building multiple layers by using the stereo-lithography, as disclosed in Japanese Patent Unexamined Publication, No. 2001-252986, as an example of forming a three dimensional structure with a liquid crystal mask (referred to as “patent reference 3”). The patent reference 3 takes advantage of the fact that the photosensitive resin becomes cured in proportion to a depth of radiated light, and it discloses a method of controlling variation of shape in a direction of depth by pre-adjusting a level of transmittancy of the radiated light with the liquid crystal mask.

Similarly, Japanese Patent Unexamined Publication, No. 1998-32160 (referred to as “patent reference 4”) discloses a method of ensuring uniformity of a size of form object within a given surface in the process of forming electronic components, wherein the size of the form object is measured and the light being radiated is corrected. This method is described more concretely as follows.

First, a produced pattern e.g., the form object is inspected with a scanning electron microscope (“SEM”) to determine a suitable amount of light exposure. Next, an amount of exposing light is measured in each area with a charge-coupled device (“CCD”) disposed above the forming work material. Finally, an amount of the light being radiated is adjusted responsive to a data taken by the measurement to ensure uniformity of the size of the form object.

The above techniques of patterning by light exposure and stereo-lithography have been used traditionally for making various types of products since they are generally suitable to form any kind of form objects comparatively easily from design data to realize a tangible model of the design. When producing electronic components by using any of the above techniques, however, it becomes necessary to adapt it to a reduced size of the form objects since overall dimensions of the form objects is about several hundreds of microns at the largest. In addition, constituent parts of the form objects further require at least another order of magnitude smaller in dimensions of the formation.

It is necessary to realize a forming accuracy of several microns, and more preferably to the level of submicron or less in order to achieve the above requirement. In this case, a planar resolution of the exposure becomes one of the factors that influence the forming accuracy. However, the planar resolution depends on whether the photosensitive resin absorbs the amount of light as designed and becomes photo-cured by a chemical reaction in predetermined areas or not. It is therefore necessary to provide an appropriate amount of light energy to the photosensitive resin at all times.

In the process of forming electronic components in particular, there are often cases that the photosensitive resin is applied to metal surfaces, e.g., conductors to ensure electrical continuity, and light is radiated to form protruding electrodes and a pattern of wiring traces, for example. In such instance, it is not only the radiated light but also the light reflected off the metal surfaces of the electronic components, for example, that contribute to the chemical reaction of the photosensitive resin. This gives rise to a problem that the form objects of the desired shape can not be produced even when a predetermined amount of light is radiated according to the design data of the form object.

For this reason, the patent reference 3 discloses the method of controlling variation of the shape in the direction of depth by pre-adjusting a level of transmittancy of the radiated light with the liquid crystal mask. Consideration has not been given in this method, however, on the effect of the reflected light from the forming work material to photo-curing of the photosensitive resin. It is therefore difficult to ensure the shape and the size as specified in design especially when forming the form objects of a predetermined shape on electrode terminals made of any metal having a large reflection factor.

Patent reference 4 discloses a method including the steps of measuring a size of the form object and correcting the light being radiated. However, this method needs to form the object first and to obtain an amount of correction in order to determine an appropriate amount of the light to be radiated. This method hence decreases the productivity since it applies the amount of correction to the form objects to be produced thereafter. It also has a problem that the form objects may not be formed uniformly and efficiently if there is a change in condition of surfaces of the forming work materials upon which the form objects are being formed by using the corrected amount of light radiation since it results in a deviation of the amount of correction from that of the originally measured forming work material.

Also disclosed is the method of achieving uniformity of the shape and dimensions by measuring the amount of exposed light in the individual areas with a CCD disposed above the forming work material and adjusting the amount of the light to be radiated according to the data taken by the measurement. The above method is effective only for the measurement of the radiated light, but it lacks consideration of the reflected light.

### SUMMARY OF THE INVENTION

The present invention relates to an electronic component forming apparatus for forming electronic components, and it has a structure comprising radiation device for radiating light to a photosensitive conductive resin provided on a forming work material, detection device for detecting reflecting light reflected from the forming work material, and control device for controlling the radiating light responsive to an amount detected by the detection device.

By virtue of this structure, the electronic component forming apparatus achieved here becomes capable of producing form objects of outstanding accuracy in shape with high productivity.

An electronic component of the present invention is formed by using the electronic component forming apparatus of this invention. It is thus possible to achieve easily the electronic component provided with protruding electrodes and a pattern of wiring traces formed thereon with outstanding accuracy in their shapes.

Furthermore, a forming method according to the present invention is a method of forming an electronic component with the electronic component forming apparatus of this invention, wherein the method comprises at least a step of radiating light to a forming work material by the radiation device and detecting reflecting light reflected from the forming work material with the detection device, a step of making a correction data for an amount of light to be radiated corresponding to a surface condition of the forming work material by making an arithmetic computing process on an amount of the reflected light, a step of applying a photosensitive conductive resin on the forming work material, a step of forming a form object of a predetermined shape by radiating light to the photosensitive conductive resin on the forming work material after adjusting the amount of light to be radiated based on the correction data, and a step of removing an unexposed portion of the photosensitive conductive resin.

This method can easily produce the electronic component provided with the form object of a predetermined shape such as protruding electrodes and a pattern of wiling traces with outstanding accuracy in their shapes.

### BRIEF DESCRIPTION OF THE DRAWINGS

FIG. 1A is a schematic diagram showing a general structure of an electronic component forming apparatus according to a first exemplary embodiment of the present invention;

FIG. 1B is a plan view showing a diagrammatic shape of a photomask used in the apparatus of FIG. 1A;

FIG. 1C is an enlarged plan view showing in detail a part of the photomask of FIG. 1B;

FIG. 2 is a flow chart illustrating a process of forming an electronic component by using the electronic component forming apparatus according to the first exemplary embodiment of this invention;

FIG. 3 is a perspective view of an electronic component produced by using the electronic component forming apparatus according to the first exemplary embodiment of the present invention;

FIG. 4A is a schematic diagram showing a general structure of an electronic component forming apparatus according to a second exemplary embodiment of the present invention;

FIG. 4B is a plan view showing a diagrammatic shape of a photomask used in the apparatus of FIG. 4A;

FIG. 4C is an enlarged plan view showing in detail a part of the photomask of FIG. 4B;

FIG. 5 is a sectional view illustrating a relation between surface condition of a semiconductor wafer and reflected light in the electronic component forming apparatus according to the second exemplary embodiment of the present invention;

FIG. 6 is a flow chart illustrating a method of forming an electronic component by using the electronic component forming apparatus according to the second exemplary embodiment of the present invention;

FIG. 7A is a schematic diagram showing a structure of an electronic component forming apparatus employing a reduced projection exposure method;

FIG. 7B is a plan view showing a diagrammatic shape of a photomask used in the apparatus of FIG. 7A; and

FIG. 7C is a plan view showing details of the photomask of FIG. 7B.

### DETAILED DESCRIPTION OF THE INVENTION

Description is provided hereinafter of exemplary embodiments of the present invention with reference to the accompanying drawings. Like reference marks are used to designate like structural components in the following descriptions and the individual drawings throughout these exemplary embodiments.

### First Exemplary Embodiment

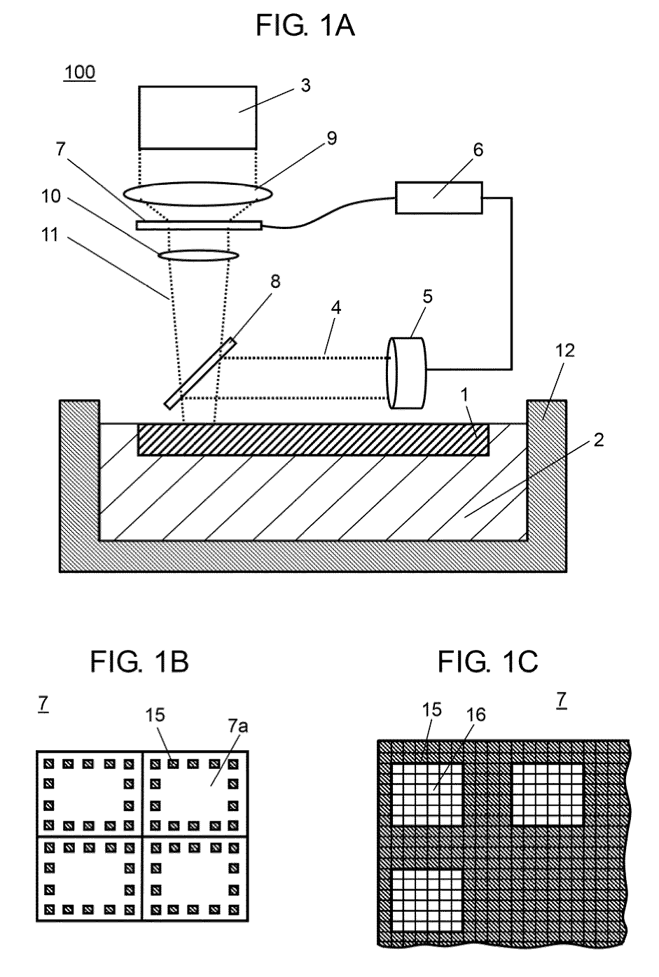

Referring now to FIG. 1A to FIG. 1C, description is provided hereinafter of an electronic component forming apparatus according to the first exemplary embodiment of this invention.

FIG. 1A is a schematic diagram showing a general structure of the electronic component forming apparatus of the first exemplary embodiment of this invention, FIG. 1B is a plan view showing a diagrammatic shape of a photomask used in the electronic component forming apparatus of FIG. 1A, and FIG. 1C is an enlarged plan view showing in detail a part of the photomask of FIG. 1B. The electronic component forming apparatus 100 of this exemplary embodiment discussed here is a device for forming a form object by exposing a photosensitive conductive resin applied on a forming work material in a predetermined thickness to light radiated according to a shape data. It is possible here to form not only a plane pattern but also a three dimensional structure with a build-up layer forming method, which is to repeat application of the photosensitive conductive resin to the forming work material and formation of a pattern by exposing it to the light. The forming work material represents any of a semiconductor element, a semiconductor wafer and a circuit board, for instance, and the form object represents any of a protruding electrode (or bump), a pattern of wiling traces and the like.

The description provided below is an example using a semiconductor wafer and a protruding electrode that are defined respectively as the forming work material and the form object.

As shown in FIG. 1A, electronic component forming apparatus 100 of this exemplary embodiment comprises at least light source 3 such as a laser, as a radiation device, lighting optical system 9, reduced-projection optical system 10, beam splitter 8, detector 5 such as a CCD, as a detection device, container 12 filled with photosensitive conductive resin 2, controller 6 as a control device, liquid crystal mask 7 and forming work material 1. In the initial stage, forming work material 1 (hereinafter referred to as “semiconductor wafer 1”) is placed in container 12 in a manner that a surface where a form object such as a protruding electrode is being formed is exposed above photosensitive conductive resin 2. It should be noted that the drawings of FIG. 1A to FIG. 1C are intended to show structures of the individual elements only schematically, and that their dimensional relation, positional relation and the like differ from the actual ones.

As shown in FIG. 1A, light 11 radiated from light source 3 is introduced into lighting optical system 9, and enters into reduced-projection optical system 10 through open segments having a pattern of given configuration formed in liquid crystal mask 7 while being adjusted by a combination of several lenses (not shown). Reduced-projection optical system 10 consisting of the combination of several lenses and a group of mirrors (not shown) brings light 11 of the pattern formed by the open segments in liquid crystal mask 7 into focus and radiates it to a predetermined area on semiconductor wafer 1. Photosensitive conductive resin 2 provided in a predetermined height (or thickness) on semiconductor wafer 1 is thus exposed to the light of the pattern of the given configuration.

In this exemplary embodiment, electronic component forming apparatus 100 of the above structure has the function of detecting reflected light 4 from an area targeted to form a protruding electrode in the following manner before semiconductor wafer 1 is provided with photosensitive conductive resin 2.

First, reflected light 4 from semiconductor wafer 1 is diverged by beam splitter 8 disposed in the optical path of light 11 and entered into detector 5, as shown in FIG. 1A.

Next, reflected light 4 entered into detector 5 is converted by detector 5 into an electrical data corresponding to an optical intensity and distribution, and the data is input to controller 6. Controller 6 then computes the input data with a given program and makes a correction data.

Based on the correction data, controller 6 controls liquid crystal cells 16 of liquid crystal mask 7 shown in FIG. 1C so as to regulate the shape or transmittancy of open segments 15. For example, controller 6 reduces a size or controls transmittancy of the open segments in a manner to decrease an amount of transmitting light in an area where an intensity of the reflected light is too high. On the other hand, controller 6 increases the size or controls transmittancy of the open segments in a manner to increase an amount of transmitting light in an area where an intensity of the reflected light is too low.

It thus becomes possible to achieve uniformity of the distribution and intensity of light 11 delivered to semiconductor wafer 1 to expose photosensitive conductive resin 2.

In this exemplary embodiment, although the description has been provided of an example of using the laser as light source 3, this is illustrative and not restrictive. This invention can be embodied with any type of light source such as a lamp and LED as long as it is capable of radiating light of a certain wavelength such as ultra-violet ray, visible light and infrared ray that can produce a chemical reaction and cure the photosensitive conductive resin.

Moreover, what has been described in this exemplary embodiment is an example of using the liquid crystal mask capable of transmitting the exposure light with a resolution equal to a number of pixels corresponding to the liquid crystal cells, but this is not restrictive. The liquid crystal mask can be replaced with any kind of device that is capable of regulating distribution of the light, as will be described later, such as a system for changing glass masks or film masks, or a shutter operated mechanically.

Furthermore, what has been described in this exemplary embodiment is an example of using the CCD as the detector. However, this is not restrictive again, and that the detector can be of any device that has a sufficiently high sensitivity for the radiated light of given wavelength and a high planar resolution. Examples of such device include a CMOS, a phototransistor, a photodiode, a photomultiplier tube and a pyroelectric detector. An additional optical magnifier may be provided in front of detector 5 to increase the detecting resolution if necessary depending on dimensions and required accuracy of the pattern formed on semiconductor wafer 1.

Moreover, although the description provided in this exemplary embodiment is an example of providing beam splitter 8 in the optical path of light 11 for diverging reflected light 4 to improve a dimensional accuracy of the pattern formed by radiation of light 11 from a perpendicular direction, this is illustrative and not restrictive. This structure may be adapted for detecting reflected light 4 directly with detector 5 without providing beam splitter 8, if for instance, light 11 is radiated at an oblique direction of a sufficiently large angle to semiconductor wafer 1. This structure can simplify the device.

In this exemplary embodiment, although the description provided is an example of making arithmetic computation of the electrical data input from the detector and controlling liquid crystal mask 7 with controller 6, this is not restrictive. Operation of the individual elements of the electronic component forming apparatus may instead be controlled altogether with controller 6. Here, a general-purpose personal computer can be used for the function of controller 6. Any computer operated according to the prescribed control program can control operation of the individual elements such as liquid crystal masks 7 and a stage for retaining a forming work material like a semiconductor wafer.

According to this exemplary embodiment, the electronic component forming apparatus controls radiation of the light by detecting the reflected light from the forming work material in advance, thereby achieving uniformity of the intensity and distribution of the light radiated to the areas to be exposed on the photosensitive conductive resin. This realizes the electronic component forming apparatus capable of forming such form objects as protruding electrodes and a pattern of wiring traces uniformly and accurately in shapes without depending on surface conditions of the forming work material. It thus becomes possible for this electronic component forming apparatus to produce electronic components having protruding electrodes and a pattern of wiring traces of uniform shapes.

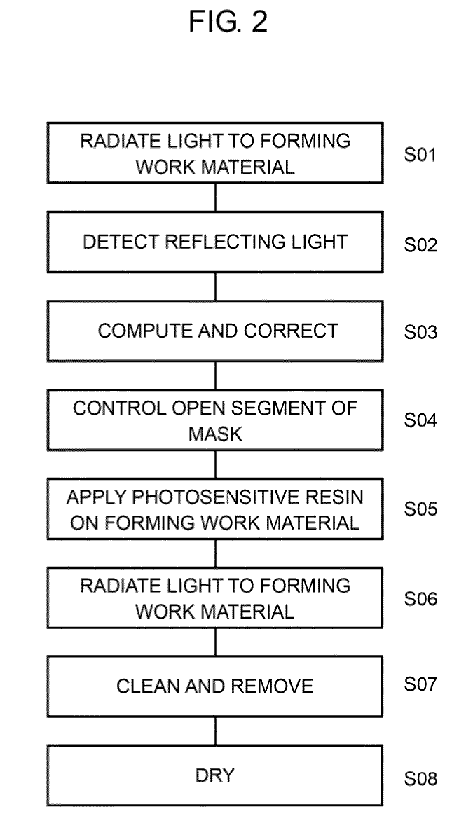

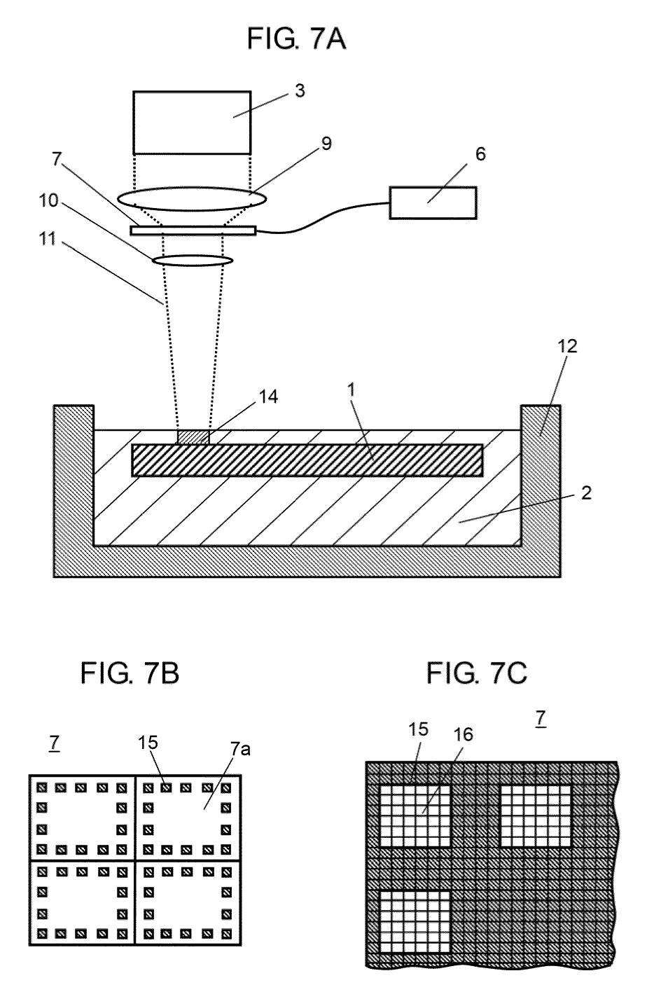

Description is provided hereinafter of a method of forming electronic component with the electronic component forming apparatus according to the first exemplary embodiment of this invention by using FIG. 2 and referring to FIG. 1A to FIG. 1C. The technique of using the reduced projection exposure method for forming electronic components provided with the protruding electrodes is similar to that described by referring to FIG. 7A to FIG. 7C, and the details will therefore be omitted here. In this method of forming electronic components, the description provided herein below will focus on a technique of controlling the radiating light in association with reflected light, as taught in this invention.

FIG. 2 is a flow chart illustrating a process of forming an electronic component by using the electronic component forming apparatus according to the first exemplary embodiment of this invention.

First, light 11 is radiated to a predetermined area to be exposed on semiconductor wafer 1, as shown in FIG. 1A (Step S01). During this step, semiconductor wafer 1 is retained in a position that an upper surface of it stays above photosensitive conductive resin 2, so that light 11 does not cause a chemical reaction of photo-cure photosensitive conductive resin 2.

Prior to the start of light radiation, the exposure pattern and semiconductor wafer 1 are checked for their positional relation, and registration is made if they are not aligned.

Reflected light 4 returning directly from semiconductor wafer 1 has a higher intensity than that traveled through the photosensitive conductive resin. It is therefore desirable to either set an output of light source 3 to a lower level or reduce an amount of light 11 that reaches semiconductor wafer 1 by adjusting the transmittancy of liquid crystal mask 7 as needed so as not to exceed a measurement range of detector 5. Although a pattern of the light radiated to semiconductor wafer 1 for detection of the reflected light is determined depending on a method of computation, as will be described later, it may be a uniform pattern having figures at regular intervals, a pattern of the actual configuration to be formed, or a uniformly radiated light on the whole surface.

Next, reflected light 4 from semiconductor wafer 1 is diverged by beam splitter 8 and detected with detector 5 comprising a CCD or the like device (Step S02). It is also practical to detect reflected light 4 directly with detector 5 without using beam splitter 8 if light 11 can be radiated at the oblique direction of a sufficiently large angle to semiconductor wafer 1, as discussed above.

In the next step, an electrical data corresponding to reflected light 4 detected by detector 5 is sent to controller 6 of liquid crystal mask 7, and it is subjected to an arithmetic computing process using the predetermined program to make a correction data (Step S03). The computing process is carried out by such method as calculating a difference from the shape defined for liquid crystal mask 7, for instance, and adding a correction factor.

Next, liquid crystal cells 16 of liquid crystal mask 7 shown in FIG. 1C is controlled responsive to the obtained correction data to regulate the shape and transmittancy of open segments 15 (Step S04). It is desirable here to repeat operation of the steps of S01 to S04 if necessary until the correction data obtained from reflected light 4 converges within a proper range.

After the correction data is made based on reflected light 4, semiconductor wafer 1 disposed on a stage (not shown) is moved down by a predetermined distance to immerse it into photosensitive conductive resin 2 in container 12, and to let the surface of exposure covered with a given thickness of photosensitive conductive resin 2 (Step S05). The photosensitive conductive resin consisting of electrically conductive filler made of metal powder such as Ag and a photosensitive resin such as a photosensitive acrylic resin may be used; As a more specific example, the photosensitive conductive resin can be comprised of a photosensitive resin having a photosensitive thermoplastic acrylic oligomer, an acrylic monomer, an initiator, a coupling agent, an adhesion additive agent, a reactive diluent, a solvent and the like, and electrically conductive filler containing 50 wt % to 95 wt % of Ag particles of a spherical form having an average diameter of 3 μm.

Besides the above immersion method, there are a number of methods for providing semiconductor wafer 1 with photosensitive conductive resin 2, such as a printing method, spin coating, die coating, spraying and dip-in method. Alternatively, a dry film may be adhered onto semiconductor wafer 1 instead of using the photosensitive conductive resin of liquid form.

Next, light 11 is radiated from light source 3 to semiconductor wafer 1 provided with photosensitive conductive resin 2 through the open segments in the liquid crystal mask having a shape and transmittancy adjusted in the step S04 responsive to the correction data (Step S06). This step photo-cures photosensitive conductive resin 2 by the light exposure, and forms a prescribed shape of form object such as protruding electrodes and a pattern of wiring traces on the predetermined area of semiconductor wafer 1.

Following the above, semiconductor wafer 1 is taken out of container 12, and the photosensitive conductive resin is cleaned to remove unexposed portions (Step S07). This cleaning process may be carried out by such technique as wet cleaning with a chemical solution, or a dry etching method such as ashing and plasma cleaning. It is desirable to use ultrasonic waves in the wet cleaning process to improve the cleaning effectiveness.

Next step of drying is carried out by a baking process at 40° C. for about one hour, for instance (Step S08). Other techniques such as air blow and nitrogen blow may also be used for this drying process.

Finally, semiconductor wafer 1 is cut and separated into individual pieces of semiconductor chips by using a dicing saw for instance.

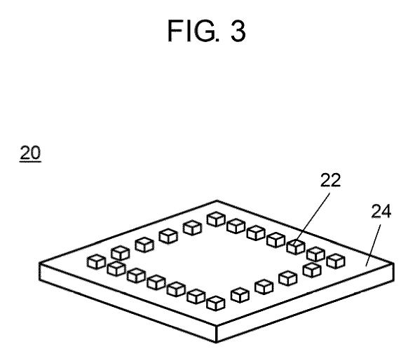

The above processes complete electronic component 20 like semiconductor chip 24 provided with the form objects of the prescribed shape such as uniformly shaped protruding electrodes 22, for example as shown in FIG. 3.

In this exemplary embodiment, what has been shown is an example of the protruding electrodes made up of a single layer, but this is not restrictive. The protruding electrodes of a multiple-layered structure can be formed by repeating the steps of S01 to S06 if necessary. When this is the case, an amount of the reflected light changes significantly in the process of forming a second layer due to surface roughness of the lower layer. It is for this reason that the method of this exemplary embodiment for correcting the intensity and distribution of the light by using the reflected light can provide an outstanding advantage of achieving a uniform shape especially when forming a multiple layer of form objects having a prescribed shape such as protruding electrodes.

Although what has been described in this exemplary embodiment is an example of using Ag as a conductive filler material of the photosensitive conductive resin, this is not restrictive. Particulate metal having an average particle size of about 0.1 μm to 10 μm made of Au, Pt, Ni, Cu, Pd, Mo or W can also be used suitably. Fine particulates of any of these metals can be used alone or in a mixed form of two or more kinds, or a powdered alloy containing any of the above metallic elements can be used as the electrically conductive filler. In addition, the conductive filler can be of any form such as a solid form, scaly form, microcrystal form, spherical form, granular form and flaky form, or it can be of an indefinite form. It is desirable, however, that the conductive filler has spherical or granular shape among these noted here. This is because such shapes provide a good optical transparency during the light exposure and to exhibit excellent exposure efficiency. The material suitable for use as the electrically conductive filler can include any one or more of solder alloys selected from the group consisting of an Sn—Ag—In alloy, Sn—Pb alloy, Sn—Ag alloy, Sn—Ag—Bi alloy, Sn—Ag—Bi—Cu alloy, Sn—Ag—In—Bi alloy, Zn—In alloy, Ag—Sn—Cu alloy, Sn—Zn—Bi alloy, In—Sn alloy, In—Bi—Sn alloy and Sn—Bi alloy. Since any of these conductive filler materials are solder alloy particulates having low melting points, they reduce degradation of the photosensitive rosin attributed to the heating temperature, for instance, when the electronic component is connected to another electronic component with the protruding electrodes formed thereon. These materials can also reduce connecting resistances since the solder alloy particulates fuse together at least partly when being connected and atoms in the electrode terminals on the mount board diffuse into the solder.

Moreover, the description provided in this exemplary embodiment is an example, in which a photosensitive acrylic resin is used as the photosensitive resin in the photosensitive conductive resin, but this is not restrictive. A photosensitive resin containing any material selected from the group consisting of a photosensitive epoxy resin, a photosensitive polyimide resin and an ene/thiol resin may be used.

According to this exemplary embodiment, the intensity and distribution of the light can be corrected before the electronic component is actually manufactured by taking into consideration an influence of the reflected light, which changes depending on the surface condition of the forming work material. This process uniformalizes the amount of light radiated to the photosensitive conductive resin on the forming work material, thereby enabling formation of the form object of the prescribed shape. As a result, the embodied apparatus becomes capable of forming the form objects such as protruding electrodes of several hundred microns or smaller, for instance, accurately and uniformly in conformity to the design data by virtue of the light radiated responsive to the correction data even if there are mixed combination of the electrodes having surfaces of a high reflectivity and a low reflectivity on the forming work material.

### Second Exemplary Embodiment

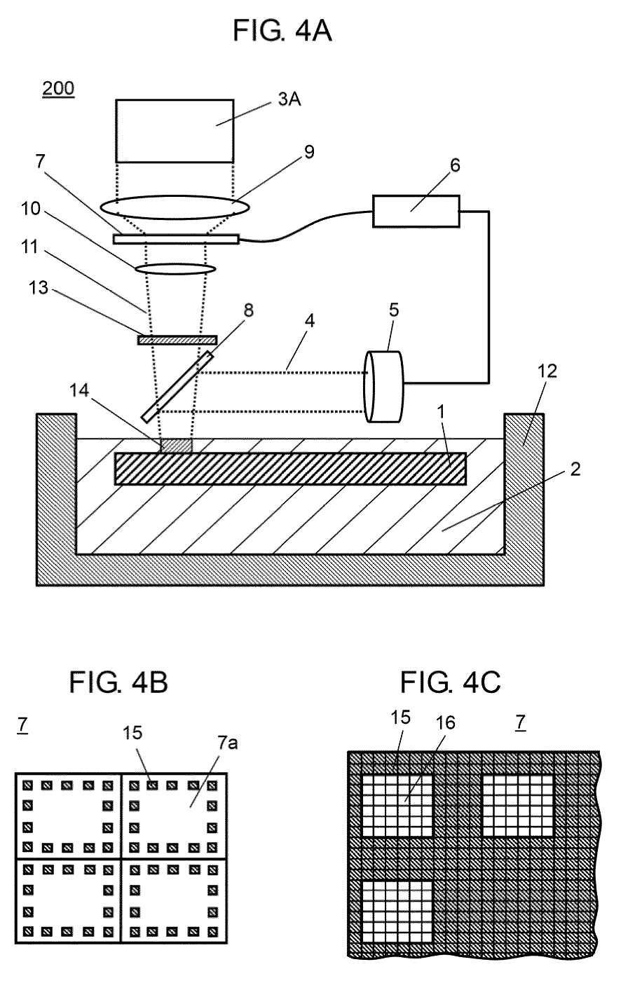

Description is provided hereinafter of an electronic component forming apparatus according to the second exemplary embodiments of the present invention with reference to FIG. 4A to FIG. 4C. Like reference marks are used throughout to designate like structural components as those of the first exemplary embodiment, and their details may be omitted.

FIG. 4A is a schematic diagram showing a general structure of the electronic component forming apparatus of the second exemplary embodiment of this invention, FIG. 4B is a plan view showing a diagrammatic shape of a photomask used in the electronic component forming apparatus of FIG. 4A, and FIG. 4C is an enlarged plan view showing in detail a part of the photomask of FIG. 4B.

Electronic component forming apparatus 200 of this exemplary embodiment comprises optical filter 13 disposed at least in a position along an optical path of light 11 between light source 3A and beam splitter 8. In addition, light source 3A differs from that of the first exemplary embodiment in respect of that it uses a halogen lamp or the like component capable of radiating at least two lights of different wavelengths. Similar components and materials as those of the first exemplary embodiment can be used for all other structural elements.

The description provided below is an example using a semiconductor wafer and a protruding electrode that are defined respectively as the forming work material and the form object in the like manner as the first exemplary embodiment.

As shown in FIG. 4A, electronic component forming apparatus 200 of this exemplary embodiment comprises at least light source 3A such as a halogen lamp, as a radiation device, lighting optical system 9, reduced-projection optical system 10, optical filter 13, beam splitter 8, detector 5 such as a CCD, as a detection device, container 12 filled with photosensitive conductive resin 2, controller 6 as a control device, liquid crystal mask 7 and forming work material 1. Under this state, forming work material 1 (hereinafter referred to as “semiconductor wafer 1”) is placed inside container 12 in a position that a surface where a form object such as a protruding electrode is being formed is covered with a given thickness (or depth) of photosensitive conductive resin 2. It should be noted here that the drawings of FIG. 4A to FIG. 4C are intended to show structures of the individual elements only schematically in the same manner as the first exemplary embodiment, and their dimensional relation, physical relation and the like differ from the actual ones.

In this embodiment, a halogen lamp or the like component capable of radiating at least two lights of different wavelengths is used suitably as light source 3A. In other words, light source 3A delivers at least the light of one wavelength for causing a chemical reaction and photo-curing the photosensitive conductive resin, and the other light of a different wavelength. To be more specific, light source 3A delivers at least the light of 500 nm or a shorter wavelength capable of photo-curing the photosensitive conductive resin when a photosensitive acrylic resin is used as the photosensitive element contained therein, and the other light of a wavelength not capable of photo-curing it.

Description is provided hereinafter of a structure of electronic component forming apparatus 200 and a correction technique according to the second exemplary embodiments of this invention.

As shown in FIG. 4A to FIG. 4C, light 11 radiated by light source 3A is delivered through lighting optical system 9, open segments 15 of liquid crystal mask 7, reduction-projection optical system 10 and optical filter 13, and radiated to semiconductor wafer 1 immersed in photosensitive conductive resin 2. Reduced-projection optical system 10 brings the light of a pattern formed by open segments 15 in liquid crystal mask 7 into focus and radiates it to a predetermined area on semiconductor wafer 1.

In this initial process, optical filter 13 cuts off the light of the wavelength having the photo-curing capability to the photosensitive resin in photosensitive conductive resin 2 while allowing the light of the other wavelength not capable of photo-curing the photosensitive resin to pass through. In this way, light 11 is radiated by light source 3A, but only of the wavelength not to photo-cure photosensitive conductive resin 2 is radiated to semiconductor wafer 1. If light source 3A delivers the light of a wavelength in the ultraviolet region capable of photo-curing a photosensitive acrylic resin and the other light of a longer wavelength, optical filter 13 suitable for use can be a high-pass filter or the like element that cuts off the light in the ultraviolet region.

Next, reflected light 4 out of light 11 of only the wavelength not to photo-cure photosensitive conductive resin 2 that passes though optical filter 13 and reflected from semiconductor wafer 1 is diverged by beam splitter 8 disposed in the optical path of light 11, and it enters detector 5, as shown in FIG. 4A.

Reflected light 4 entered in detector 5 is converted by detector 5 into an electrical data corresponding to its optical intensity and distribution, and the data is input to controller 6. Controller 6 then carries out a computing process of the input data with a prescribed program and makes a correction data. It is desirable here to repeat the above steps as needed until the correction data obtained from reflected light 4 converges within a proper range.

Based on the correction data, controller 6 controls liquid crystal cells 16 of liquid crystal mask 7 shown in FIG. 4C so as to regulate the shape and transmittancy of open segments 15. For example, controller 6 reduces a size or controls transmittancy of the open segments in a manner to decrease an amount of transmitting light in an area where an intensity of the reflected light is too high. On the other hand, controller 6 increases the size or controls transmittancy of the open segments in a manner to increase an amount of transmitting light in an area where an intensity of the reflected light is too low. It thus becomes possible to achieve uniformity of the distribution and intensity of light 11 delivered to semiconductor wafer 1 to expose photosensitive conductive resin 2.

After the correction data is made, optical filter 13 is moved out of the optical path of light 11 by a drive system, which is not shown in the drawings. This allows the light radiated by light source 3A of the wavelength capable of photo-curing the photosensitive resin contained in photosensitive conductive resin 2 to be radiated to photosensitive conductive resin 2 of a given height (or thickness) covering semiconductor wafer 1. The light exposes photosensitive conductive resin 2 in a prescribed pattern to form a form object of such configuration as protruding electrodes and a pattern of wiling traces on semiconductor wafer 1. Although light source 3A also radiates the light of the wavelength not contributing to photo-curing of photosensitive conductive resin 2 at the same time, this does not pose any problem since it has no effect of photo-curing to the photosensitive conductive resin.

In this exemplary embodiment, although the description provided is an example of disposing optical filter 13 in the posterior to reduced-projection optical system 10, this is illustrative and not restrictive. Optical filter 13 can be disposed in any position along the optical path anterior to where light 11 reaches photosensitive conductive resin 2 on semiconductor wafer 1.

Moreover, what has been described in this exemplary embodiment is an example comprising light source 3A of a halogen lamp, but again this is not restrictive. Light source 3A may be composed of two or more sets of single wave laser or lamp, each of which radiates light of the wavelength capable of causing a chemical reaction and photo-curing the photosensitive resin in the photosensitive conductive resin, and light of other wavelengths. When this is the case, the individual light sources can be used selectively by switching them with switch device such as an optical shutter or a mechanical system. Some examples usable as the mechanical system include a slider for switching the light sources or their optical paths, a mechanical shutter for switching the optical paths, and a mirror for switching the optical paths.

According to this exemplary embodiment, use of the optical filter makes the electronic component forming apparatus capable of detecting the reflected light even though the photosensitive conductive resin covers the forming work material, and controlling radiation of the light, to thereby achieve uniformity of the intensity and distribution of the light radiated to the areas to be exposed on the photosensitive conductive resin. This realizes the electronic component forming apparatus capable of forming such form objects as protruding electrodes and a pattern of wiling traces uniformly and accurately in their shapes without depending on the surface condition of the forming work material. It thus becomes possible for this electronic component forming apparatus to produce electronic components having protruding electrodes and a pattern of wiring traces of uniform shapes. This forming apparatus also improves productivity since the correction data can be made while the photosensitive conductive resin remains covering the upper surface of the semiconductor wafer.

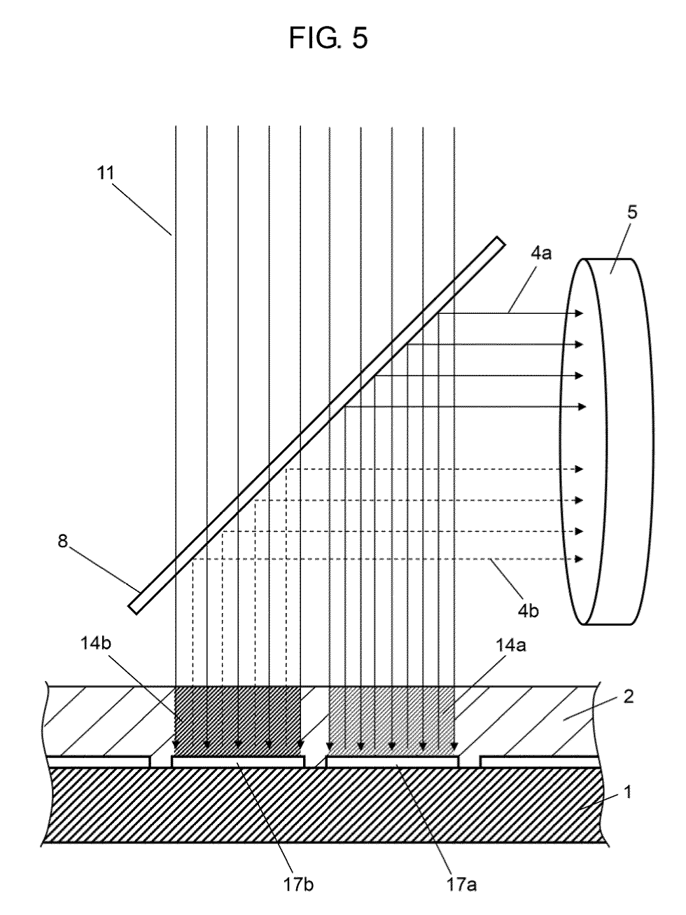

Referring now to FIG. 5, description is given hereinafter of a relation between surface condition of the semiconductor wafer and reflected light in the second exemplary embodiment of this invention.

FIG. 5 is a sectional view illustrating the relation between surface condition of the semiconductor wafer and reflected light in the electronic component forming apparatus according to the second exemplary embodiment of this invention.

Light 11 radiated from the light source passes through photosensitive conductive resin 2, and it is reflected off the surface of semiconductor wafer 1 as shown in FIG. 5. In general, semiconductor wafer 1 is provided on its surface with a number of electrode terminals formed of different metallic materials such as Al and Au, or electrode terminals of different reflectivity due to difference in the surface conditions attributed to stains, etc. even if they are formed of the same material. This results in variations in value of the individual amounts of reflected lights 4 as they are reflected off the various surfaces. When there are electrodes 17a formed of Al exhibiting high reflectivity on their clean surfaces and electrodes 17b also formed of Al but exhibiting low reflectivity on their stained surfaces, for instance, reflected light 4a from electrodes 17a of high reflectivity shows a higher intensity than reflected light 4b from electrodes 17b of low reflectivity even if light 11 of the same amount of light is radiated in the same pattern. Photosensitive conductive resin 2 is exposed to the light of an amount given by the multiplication of the radiated light and the reflected light. A portion of photosensitive conductive resin 2 in area 14a where the reflected light is stronger is therefore exposed to a greater amount of the light than another portion of photosensitive conductive resin 2 in area 14b where the reflected light is weaker. In other words, photosensitive conductive resin 2 in area 14b where the reflected light is weaker becomes underexposed when the amount of light is set properly for exposing photosensitive conductive resin 2 in area 14a where the reflected light is stronger. On the other hand, photosensitive conductive resin 2 in area 14a where the reflected light is stronger becomes overexposed when the amount of light is set properly for exposing photosensitive conductive resin 2 in area 14b where the reflected light is weaker. As a result, sizes of the form objects such as protruding electrodes located in the overexposed area become larger even if semiconductor wafer 1 is radiated evenly with the light of equal amount.

To this end, the present exemplary embodiment uses the light of one wavelength that passes through the optical filter and incapable of exposing the photosensitive conductive resin for prior detection of the intensity and distribution of the amount of the reflected light with detector 5, among the light of different wavelengths radiated from the light source. Using the detected data, an arithmetic computation is made first to obtain a difference, and this connection data is used to regulate the shape and transmittancy of open segments of the liquid crystal mask. This thus makes possible to equalize the amount of light for exposing the photosensitive conductive resin and produce form objects of uniform configuration without regard to the surface condition of the semiconductor wafer.

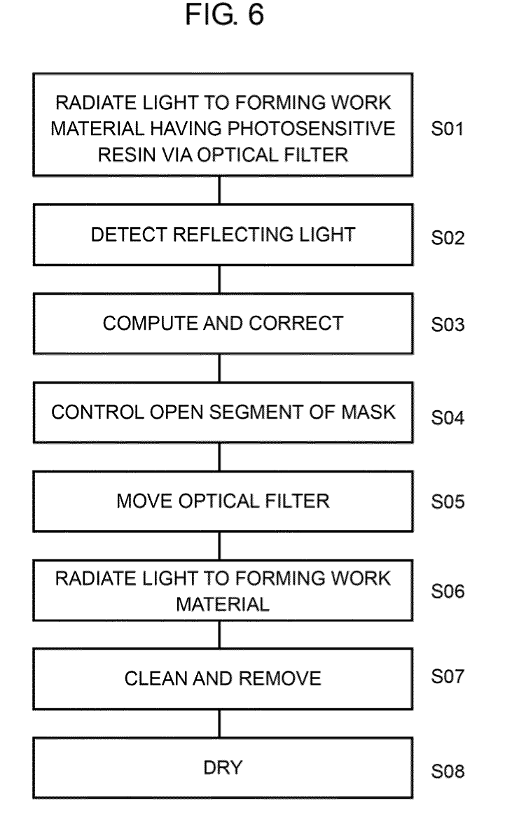

Description is provided hereinafter of a method of forming electronic component with the electronic component forming apparatus according to the second exemplary embodiment of this invention by using FIG. 6 and referring to FIG. 4A to FIG. 4C. The technique of using the reduced projection exposure method for forming electronic components provided with the protruding electrodes is similar to that already described with reference to FIG. 7A to FIG. 7C, and the details will therefore be omitted here. In this exemplary embodiment, a method of detecting the reflected light is the only aspect different from the first exemplary embodiment. Other processes of the forming method will be touched only briefly because of their similarity.

FIG. 6 is a flow chart illustrating a process of forming an electronic component by using the electronic component forming apparatus according to the second exemplary embodiment of this invention.

First, semiconductor wafer 1 is retained in a immersed position of a predetermined depth from the surface of photosensitive conductive resin 2 contained in container 12, as shown in FIG. 4A. Under this condition, light 11 consisting of at least two different wavelengths from light source 3A is radiated to given area 14 to be exposed on semiconductor wafer 1 through optical filter 13 disposed in the optical path (Step S01). At this moment, the light of the wavelength not capable of exposing photosensitive conductive resin 2 among lights 11 radiated by light source 3A is radiated to semiconductor wafer 1 after passing through optical filter 13. That is, the light of the wavelength capable of exposing photosensitive conductive resin 2 among lights 11 radiated by light source 3A is cut off by optical filter 13. Photosensitive conductive resin 2 on semiconductor wafer 1 is therefore not exposed with the light.

Next, reflected light 4 of the wavelength not capable of exposing photosensitive conductive resin 2 reflected from semiconductor wafer 1 is diverged by beam splitter 8, so that it is detected with detector 5 comprising a CCD or the like device (Step S02).

In the next step, an electrical data corresponding to reflected light 4 detected by detector 5 is sent to controller 6 of liquid crystal mask 7, and it is subjected to an arithmetic computing process using the predetermined program to make a correction data (Step S03). The computing process is carried out by such method as calculating a difference from the shape defined for liquid crystal mask 7, for instance, and adding a correction factor.

Next, liquid crystal cells 16 of liquid crystal mask 7 shown in FIG. 4C is controlled responsive to the obtained correction data to regulate the shape and transmittancy of open segments 15 (Step S04). It is desirable here to repeat operation of the steps of S01 to S04 if necessary until the correction data obtained from reflected light 4 converges within a proper range. The correction data is thus made based on reflected light 4.

In the next step, optical filter 13 is moved out of the optical path of light 11 radiated from light source 3A (Step S05). The photosensitive conductive resin remains in the same state as that of the first exemplary embodiment at this time.

Next, light 11 is radiated from light source 3A to semiconductor wafer 1 provided with photosensitive conductive resin 2 through the open segments in the liquid crystal mask having a shape and transmittancy adjusted in the step S04 responsive to the correction data (Step S06). This step cures photosensitive conductive resin 2 by the light of the wavelength capable of exposing photosensitive conductive resin 2 among those radiated from light source 3A. This step forms a prescribed shape of form object such as protruding electrodes and a pattern of wiring traces on predetermined area 14 of semiconductor wafer 1.

Following the above, semiconductor wafer 1 is taken out of container 12, and photosensitive conductive resin 2 is cleaned to remove the unexposed portions (Step S07). This cleaning process may be carried out by such technique as wet cleaning with a chemical solution, or a dry etching method such as ashing and plasma cleaning. It is desirable to use ultrasonic waves in the wet cleaning process to improve the cleaning effectiveness.

Next step of drying is carried out by a baking process at 40° C. for about one hour, for instance (Step S08). Other techniques such as air blow and nitrogen blow may also be used for the drying process.

Finally, semiconductor wafer 1 is cut and separated into individual pieces of semiconductor chips by using a dicing saw, for instance.

The above processes complete electronic component 20 like semiconductor chip 24 provided with the form objects of the prescribed shape such as uniformly shaped protruding electrodes 22, for example as shown in FIG. 3 in the first exemplary embodiment.

In this exemplary embodiment, what has been shown is an example of the protruding electrodes made up of a single layer, but this is not restrictive. The protruding electrodes of a multiple-layered structure can be formed by repeating the steps of S01 to S06 if necessary. When this is the case, an amount of the reflected light changes significantly in the process of forming a second layer due to surface roughness of the lower layer. It is for this reason that the method of this exemplary embodiment for correcting the intensity and distribution of the light by using the reflected light can provide an outstanding advantage of achieving a uniform shape especially when forming a multiple layer of form objects having a prescribed shape such as the protruding electrodes.

Although what has been described in this exemplary embodiment is an example of making the correction data and carrying out the control function by using the reflected light while the forming work material is covered with the photosensitive conductive resin, this is illustrative and not restrictive. The detection of the reflected light and the control function may be carried out with the forming work material exposed for instance, in the same manner as the first exemplary embodiment.

Moreover, although the description provided in this exemplary embodiment is an example having the optical filter for cutting off the light of the wavelength capable of photo-curing the photosensitive conductive resin, this is also illustrative and not restrictive. An optical filter with the function of switching selectively between the light of photo-curing wavelength and another light of not photo-curing wavelength can be used. Such an optical filter helps form the form objects of a sharp and uniform configuration even if the light has a wide range of not photo-curing wavelength.

Furthermore, the description provided in this exemplary embodiment is an example of using the light source for radiating the light of at least two different wavelengths capable of exposing the photosensitive conductive resin and not capable of exposing the photosensitive conductive resin, but this is not restrictive. The electronic component forming apparatus may be so embodied as to comprise two light sources for radiating the light of the wavelength capable of exposing the photosensitive conductive resin and another wavelength capable of exposing the photosensitive conductive resin respectively, so that these light sources can be switched for lighting. Use of this structure can eliminate the optical filter, and the simplified apparatus can improve productivity for producing electronic components.

What is claimed is:

1. An electronic component forming apparatus for forming an electronic component, the apparatus comprising:
radiation device for radiating light to a photosensitive conductive resin provided on a forming work material;
detection device for detecting reflecting light reflected from the forming work material; and
control device for controlling the radiating light responsive to an amount detected by the detection device.

2. The electronic component forming apparatus of claim 1, wherein the radiation device comprises a light source for radiating light having at least two different wavelengths.

3. The electronic component forming apparatus of claim 2, wherein the light source radiates at least light of a wavelength capable of exposing the photosensitive conductive resin and a wavelength not capable of exposing the photosensitive conductive resin.

4. The electronic component forming apparatus of claim 3 further comprising switch device disposed between the radiation device and the photosensitive conductive resin for switching between the light of the wavelength capable of exposing the photosensitive conductive resin and the light of the wavelength not capable of exposing the photosensitive conductive resin.

5. The electronic component forming apparatus of claim 1 wherein the detection device uses at least light of a wavelength not capable of exposing the photosensitive conductive resin within the reflected light that reflects from the forming work material.

6. An electronic component comprising a forming work material provided with a form object produced by the electronic component forming apparatus of claim 1.

7. A method of forming an electronic component by using the electronic component forming apparatus of claim 1, the method comprising:
radiating the light to a forming work material by radiation device and detecting reflecting light reflected from the forming work material with detection device;
making a correction data for an amount of light to be radiated corresponding to a surface condition of the forming work material by making an arithmetic computing process using an amount of the reflected light;
applying a photosensitive conductive resin on the forming work material;
forming a form object of a predetermined shape by radiating light to the photosensitive conductive resin on the forming work material after adjusting an amount of the light to be radiated based on the correction data; and
removing an unexposed portion of the photosensitive conductive resin.

8. The method of forming an electronic component of claim 7, wherein adjusting an amount of the light is carried out by regulating shape and transmittancy of an open segment in a liquid crystal mask.

9. The method of forming an electronic component of claim 7, wherein the radiation device comprises a light source for radiating light having at least two different wavelengths.

10. The method of forming an electronic component of claim 9, wherein the light source radiates at least light of a wavelength capable of exposing the photosensitive conductive resin and a wavelength not capable of exposing the photosensitive conductive resin.

11. The method of forming an electronic component of claim 10, wherein radiating light is carried out by switching the light between one wavelength capable of exposing the photosensitive conductive resin and the wavelength not capable of exposing the photosensitive conductive resin in a position between the radiation device and the photosensitive conductive resin.

12. The method of forming an electronic component of claim 7, wherein the detection device uses at least light of a wavelength not capable of exposing the photosensitive conductive resin among the reflected light that reflects from the forming work material.
